(19) United States
(12) Patent Application Publication
(10) Pub. No.: US 2014/0037701 A1
(43) Pub. Date: Feb. 6, 2014

(54) STACKED LOTIONED AND FOLDED WEB SUBSTRATES

(71) Applicants: André Mellin, Amberley Village, OH (US); Monica Ho-Kleinwaechter, Loveland, OH (US); Joseph L. Cuellar, Independence, KY (US)

(72) Inventors: André Mellin, Amberley Village, OH (US); Monica Ho-Kleinwaechter, Loveland, OH (US); Joseph L. Cuellar, Independence, KY (US)

(21) Appl. No.: 13/567,491

(22) Filed: Aug. 6, 2012

(51) Int. Cl. A61K 8/02; A61K 8/36; A61Q 19/00; A61K 8/37; A61K 8/31; A61K 8/34; A61K 8/42

(52) U.S. Cl. 424/401; 514/724; 514/558; 514/738; 514/625; 514/552; 514/762

(57) ABSTRACT

A stack of folded web substrates is disclosed. Each folded web substrate has a folded surface area of about A/8 produced from a respective unfolded web substrate having an unfolded surface area of A and having a lotion applied to one surface thereof. The stack of folded web substrates has a normalized Mellin gauge value of greater than 0.06.

### FIELD OF THE INVENTION

The present disclosure generally relates to stacks of folded web substrates such as tissue paper products. More specifically, the present disclosure relates to stacks of folded lotioned tissue paper products.

### BACKGROUND OF THE INVENTION

Facial tissue is sold in a variety of packages, including a small plastic film package commonly referred to as a pocket pack. These packages are convenient for keeping in pockets, purses, automobile glove compartments, and the like where the larger tissue cartons would be inconvenient or impossible to keep. Some pocket pack packages may include a re-sealable opening to protect the unused tissues after the package has been opened. The package opening is usually created by providing perforations in one of the package sidewalls to define a flap to cover the opening when the perforations are broken. An exemplary package is discussed in U.S. Pat. No. 4,460,088.

It is desirable for certain facial tissues to be soft and lubricious. Softness is a complex tactile impression elicited by a product when it is stroked against the skin. The purpose of being soft and lubricious is so that these products can be used to cleanse the skin without being irritating. Objectionable otorhinolaryngogical discharges do not always occur at a time and place convenient for one to perform a thorough cleansing, as with soap and copious amounts of water. A wide variety of tissue and toweling products are offered to aid in the task of removing from the skin and retaining the before mentioned discharges for disposal in a sanitary fashion. Not surprisingly, the use of these products does not approach the level of cleanliness and soothing that can be achieved by the more thorough cleansing methods. Thus, producers of facial tissue products are constantly striving to make their products compete more favorably with thorough cleansing methods on a level par with the ability and need to sooth.

One of the most important physical properties related to softness is generally considered by those skilled in the art to be the strength of the web. Strength is the ability of the product, and its constituent webs, to maintain physical integrity and to resist tearing, bursting, and shredding under use conditions. Achieving a high softening potential without degrading strength has long been an object of workers in the field of the present invention. Accordingly, making soft tissue products which promote comfortable cleaning without performance impairing sacrifices has long been the goal of the engineers and scientists who are devoted to research into improving tissue paper. There have been numerous attempts to reduce the abrasive effect, i.e., improve the softness of tissue products of facial tissue sold in pocket packs.

One area that has been exploited in this regard has been to select and modify cellulose fiber morphologies and engineer paper structures to take optimum advantages of the various available morphologies. Applicable art in this area include in U.S. Pat. Nos. 5,228,954; 5,405,499; 4,874,465; and 4,300,981. Another area which has received a considerable amount of attention is the addition of chemical softening agents (also referred to herein as “chemical softeners”) to tissue and toweling products. Applicable art in this area include in U.S. Pat. Nos. 5,215,626; 5,246,545; and 5,525,345.

However, there is a void of facial tissue products sold in pocket packs that are both soft and provide a lotion or emollient to sooth the otorhinolaryngogical area after cleaning. Accordingly, it would be desirable to be able to provide a lubricious, strong, and yet soft facial tissue that is packaged in a pocket pack that is convenient for keeping in pockets, purses, automobile glove compartments, and the like.

### SUMMARY OF THE INVENTION

The present disclosure provides for a stack of folded web substrates. Each folded web substrate has a folded surface area of about A/8 produced from a respective unfolded web substrate having an unfolded surface area of A and having a lotion applied to one surface thereof. The stack of folded web substrates has a normalized Mellin gauge value of greater than 0.06.

The present disclosure also provides for a stack of folded web substrates where each has a folded surface area of about A/8. Each folded web substrate is produced from a respective un-folded web substrate having an unfolded surface area of A and having a lotion applied to one surface thereof. The stack of folded web substrates has a normalized Mellin gauge value satisfying the equation y≧−0.008 ln(x)+0.1297 where x=applied mass and y=normalized Mellin Gauge (by caliper) value.

### BRIEF DESCRIPTION OF THE DRAWINGS

FIG. 1 is a perspective view of an exemplary pocket pack for containing folded and stacked lotioned facial tissues;

FIG. 2 is a series of plan views of an individual facial tissue in all of its sequential folding configurations as it is folded into a size and shape preferable for a pocket package;

FIG. 3 is a cross sectional view of the finally folded facial tissue of FIG. 2 taken along line 3-3;

FIG. 4 is a perspective view of a stack of finally folded facial tissues; and,

FIG. 5 is a graphical representation of the thickness of facial tissue packages of the present disclosure (Mellin gauge) to caliper of the facial tissue (normalized for sheet count differences) vs. applied mass.

### DETAILED DESCRIPTION OF THE INVENTION

As used herein, the term “water soluble” refers to materials that are soluble in water to at least 3%, by weight, at 25° C.

As used herein, the terms “tissue paper web,” “paper web,” “web,” “paper sheet,” “tissue paper,” “tissue product,” “facial tissue,” “web substrate,” and “paper product” are all used interchangeably to refer to sheets of paper made by a process comprising the steps of forming an aqueous papermaking furnish, depositing this furnish on a foraminous surface, such as a Fourdrinier wire, and removing the water from the furnish (e.g., by gravity or vacuum-assisted drainage), forming an embryonic web, transferring the embryonic web from the forming surface to a transfer surface traveling at a lower speed than the forming surface. The web is then transferred to a fabric upon which it is through air dried to a final dryness after which it is wound upon a reel.

The terms “multi-layered tissue paper web,” “multi-layered paper web,” “multi-layered web,” “multi-layered paper sheet,” and “multi-layered paper product” are all used interchangeably to refer to sheets of paper prepared from two or more layers of aqueous paper making furnish. The layers are preferably formed from the deposition of separate streams of dilute fiber slurries upon one or more endless foraminous surfaces. If the individual layers are initially formed on separate foraminous surfaces, the layers can be subsequently combined when wet to form a multi-layered tissue paper web.

As used herein, the term “chemical softening agent” refers to any chemical ingredient which improves the tactile sensation perceived by the consumer that holds a particular paper product and rubs it across the skin. Softness is a particularly important property for facial tissues. Such tactile perceivable softness can be characterized by, but is not limited to, friction, flexibility, and smoothness, as well as subjective descriptors, such as lubricious, velvet, silk or flannel, which imparts a lubricious feel to tissue. This includes, for exemplary purposes only, polyhydroxy compounds.

As used herein, the term “single-ply tissue product” means that it is comprised of one ply of creped or un-creped tissue; the ply can be substantially homogeneous in nature or it can be a multi-layered tissue paper web.

As used herein, the term “multi-ply tissue product” means that it is comprised of more than one ply of creped or uncreped tissue. The plies of a multi-ply tissue product can be substantially homogeneous in nature or they can be multi-layered tissue paper webs.

As used herein, the term “polyhydroxy compound” is defined as a chemical agent that imparts lubricity or emolliency to tissue paper products and also possesses permanence with regard to maintaining the fidelity of its deposits without substantial migration when exposed to the environmental conditions to which products of this type are ordinarily exposed during their typical life cycle. The present invention contains as an essential component from about 2.0% to about 30.0%, preferably from 5% to about 20.0%, more preferably from about 8.0% to about 15.0%, of a water soluble polyhydroxy compound based on the dry fiber weight of the tissue paper. In another embodiment, the present invention may contain as an essential component an application of from about 0.1 g/m² to about 36 g/m², preferably from about 0.55 g/m² to about 20 g/m² more preferably from about 0.65 g/m² to about 12 g/m², of a water soluble polyhydroxy compound to the tissue paper.

Examples of water soluble polyhydroxy compounds suitable for use in the present invention include glycerol, polyglycerols having a weight average molecular weight of from about 150 to about 800 and polyoxyethylene and polyoxypropylene having a weight-average molecular weight of from about 200 to about 4000, preferably from about 200 to about 1000, most preferably from about 200 to about 600. Polyoxyethylene having a weight average molecular weight of from about 200 to about 600 are especially preferred. Mixtures of the above-described polyhydroxy compounds may also be used. For example, mixtures of glycerol and polyglycerols, mixtures of glycerol and polyoxyethylenes, ‘mixtures of polyglycerols and polyoxyethylenes, etc. are useful in the present invention. A particularly preferred polyhydroxy compound is polyoxyethylene having a weight average molecular weight of about 200. This material is available commercially from the BASF Corporation of Florham Park, N.J. under the trade names “Pluriol E200” and “Pluracol E200”.

As used herein, the term “lotion” is defined as an oil, emollient, wax, and/or immobilizing agent intended for external application to a surface that can be adapted to contain agents for soothing or softening the skin, such as that of the face or hands. In one example, the lotion composition comprises from about 10% to about 90% and/or from about 30% to about 90% and/or from about 40% to about 90% and/or from about 40% to about 85% of an oil, wax, and/or emollient. In another example, the lotion composition comprises from about 10% to about 50% and/or from about 15% to about 45% and/or from about 20% to about 40% of an immobilizing agent. In another example, the lotion composition comprises from about 0% to about 60% and/or from about 5% to about 50% and/or from about 5% to about 40% of petrolatum.

Lotion compositions of the present invention may be heterogeneous. They may contain solids, gel structures, polymeric material, a multiplicity of phases (such as oily and water phase) and/or emulsified components. It may be difficult to determine precisely the melting temperature of the lotion composition (i.e. difficult to determine the temperature of transition between the liquid form, the quasi-liquid form, the quasi-solid form, and the solid form). The terms melting temperature, melting point, transition point and transition temperature are used interchangeably in this document and have the same meaning. The lotion can be applied to a substrate in combination with other additives including, but not limited to, polyhydroxy compounds. As one of skill in the art would recognize, a lotion of the present invention may be combined with a polyhydroxy compound of the present invention and applied to the surface of a tissue paper web of the present invention as a mixture, or may be applied to a tissue paper web neat followed by an application of a polyhydroxy compound. Alternatively, as would be known to one of skill in the art, a polyhydroxy compound may be applied to the surface of a tissue paper web neat followed by an application of a lotion.

The lotion compositions may be semi-solid, of high viscosity so they do not substantially flow without activation during the life of the product or gel structures. The lotion compositions may be shear thinning and/or they may strongly change their viscosity around skin temperature to allow for transfer and easy spreading on a user's skin. Additionally, the lotion compositions may be in the form of emulsions and/or dispersions.

In one example of a lotion composition, the lotion composition has a water content of less than about 20% and/or less than 10% and/or less than about 5% or less than about 0.5%. In another example, the lotion composition may have a solids content of at least about 15% and/or at least about 25% and/or at least about 30% and/or at least about 40% to about 100% and/or to about 95% and/or to about 90% and/or to about 80%.

A non-limiting example of a suitable lotion composition of the present invention comprises a chemical softening agent, such as oil and/or emollient, that softens, soothes, supples, coats, lubricates, or moisturizes the skin. The lotion composition may sooth, moisturize, and/or lubricate a user's skin. Non-limiting examples of suitable oils and/or emollients include glycols (such as propylene glycol and/or glycerine), polyglycols (such as triethylene glycol), petrolatum, fatty acids, fatty alcohols, fatty alcohol ethoxylates, fatty alcohol esters and fatty alcohol ethers, fatty acid ethoxylates, fatty acid amides and fatty acid esters, hydrocarbon oils (such as mineral oil), squalane, fluorinated emollients, silicone oil (such as dimethicone) and mixtures thereof. Non-limiting examples of emollients useful in the present invention can be petroleum-based, fatty acid ester type, alkyl ethoxylate type, or mixtures of these materials. Suitable petroleum-based emollients include those hydrocarbons, or mixtures of hydrocarbons, having chain lengths of from 16 to 32 carbon atoms. Petroleum based hydrocarbons having these chain lengths include petrolatum (also known as “mineral wax,” “petroleum jelly” and “mineral jelly”). Petrolatum usually refers to more viscous mixtures of hydrocarbons having from 16 to 32 carbon atoms. A suitable Petrolatum is available from Witco, Corp., Greenwich, Conn. as White Protopet® 1 S.

Suitable fatty acid ester emollients include those derived from long chain C₁₂-C₂₈ fatty acids, such as C₁₆-C₂₂ saturated fatty acids, and short chain C₁-C₈ monohydric alcohols, such as C₁-C₃ monohydric alcohols. Non-limiting examples of suitable fatty acid ester emollients include methyl palmitate, methyl stearate, isopropyl laurate, isopropyl myristate, isopropyl palmitate, and ethylhexyl palmitate. Suitable fatty acid ester emollients can also be derived from esters of longer chain fatty alcohols (C₁₂-C₂₈, such as C₁₂-C₁₆) and shorter chain fatty acids e.g., lactic acid, such as lauryl lactate and cetyl lactate.

Suitable alkyl ethoxylate type emollients include C₁₂-C₁₈ fatty alcohol ethoxylates having an average of from 3 to 30 oxyethylene units, such as from about 4 to about 23 oxyethylene units. Non-limiting examples of such alkyl ethoxylates include laureth-3 (a lauryl ethoxylate having an average of 3 oxyethylene units), laureth-23 (a lauryl ethoxylate having an average of 23 oxyethylene units), ceteth-10 (acetyl ethoxylate having an average of 10 oxyethylene units), steareth-2 (a stearyl ethoxylate having an average of 2 oxyethylene units) and steareth-10 (a stearyl ethoxylate having an average of 10 oxyethylene units). These alkyl ethoxylate emollients are typically used in combination with the petroleum-based emollients, such as petrolatum, at a weight ratio of alkyl ethoxylate emollient to petroleum-based emollient of from about 1:1 to about 1:3, preferably from about 1:1.5 to about 1:2.5.

The lotion compositions of the present invention may include an “immobilizing agent.” Without desiring to be bound by theory, it is believed that immobilizing agents are believed to prevent migration of the emollient so that it can remain primarily on the surface of the fibrous structure to which it is applied. In this way, the emollient may deliver maximum softening benefit as well as be available for transferability to the user's skin. Suitable immobilizing agents for the present invention can comprise polyhydroxy fatty acid esters, polyhydroxy fatty acid amides, and mixtures thereof. To be useful as immobilizing agents, the polyhydroxy moiety of the ester or amide should have at least two free hydroxy groups. It is believed that these free hydroxy groups are the ones that co-crosslink through hydrogen bonds with the cellulosic fibers of the tissue paper web to which the lotion composition is applied and homo-crosslink, also through hydrogen bonds, the hydroxy groups of the ester or amide, thus entrapping and immobilizing the other components in the lotion matrix. Non-limiting examples of suitable esters and amides will have three or more free hydroxy groups on the polyhydroxy moiety and are typically nonionic in character. Because of the skin sensitivity of those using paper products to which the lotion composition is applied, these esters and amides should also be relatively mild and non-irritating to the skin.

Immobilizing agents include agents that are may prevent migration of the emollient into the fibrous structure such that the emollient remain primarily on the surface of the fibrous structure and/or sanitary tissue product and/or on the surface treating composition on a surface of the fibrous structure and/or sanitary tissue product and facilitate transfer of the lotion composition to a user's skin. Immobilizing agents may function as viscosity increasing agents and/or gelling agents.

Non-limiting examples of suitable immobilizing agents include waxes (such as ceresin wax, ozokerite, microcrystalline wax, petroleum waxes, fisher tropsh waxes, silicone waxes, paraffin waxes), fatty alcohols (such as cetyl, cetaryl, cetearyl and/or stearyl alcohol), fatty acids and their salts (such as metal salts of stearic acid), mono and polyhydroxy fatty acid esters, mono and polyhydroxy fatty acid amides, silica and silica derivatives, gelling agents, thickeners and mixtures thereof. In one example, the lotion composition comprises at least one immobilizing agent and at least one emollient.

One or more skin benefit agents may be included in the lotion composition of the present disclosure. If a skin benefit agent is included in the lotion composition, it may be present in the lotion composition at a level of from about 0.5% to about 80% and/or 0.5% to about 70% and/or from about 5% to about 60% by weight of the lotion. Non-limiting examples of skin benefit agents include zinc oxide, vitamins, such as Vitamin B3 and/or Vitamin E, sucrose esters of fatty acids, such as Sefose 16185 (commercially available from Procter & Gamble Chemicals), antiviral agents, anti-inflammatory compounds, lipid, inorganic anions, inorganic cations, protease inhibitors, sequestration agents, chamomile extracts, aloe vera, calendula officinalis, alpha bisalbolol, Vitamin E acetate and mixtures thereof.

Non-limiting examples of suitable skin benefit agents include fats, fatty acids, fatty acid esters, fatty alcohols, triglycerides, phospholipids, mineral oils, essential oils, sterols, sterol esters, emollients, waxes, humectants and combinations thereof. In one example, the skin benefit agent may be any substance that has a higher affinity for oil over water and/or provides a skin health benefit by directly interacting with the skin. Suitable examples of such benefits include, but are not limited to, enhancing skin barrier function, enhancing moisturization and nourishing the skin. The skin benefit agent may be alone, included in a lotion composition and/or included in a surface treating composition.

The lotion composition may be a transferable lotion composition. A transferable lotion composition comprises at least one component that is capable of being transferred to an opposing surface such as a user's skin upon use. In one example, at least 0.1% of the transferable lotion present on the user contacting surface transfers to the user's skin during use.

Other optional ingredients that may be included in the lotion composition include vehicles, perfumes, especially long lasting and/or enduring perfumes, antibacterial actives, antiviral actives, disinfectants, pharmaceutical actives, film formers, deodorants, opacifiers, astringents, solvents, and cooling sensate agents such as camphor, thymol and menthol.

EXEMPLARY LOTION COMPOSITION #1 (nominal w/w)

| | |
|---|---|
| Stearyl Alcohol CO1897 | 40% w/w |
| Petrolatum Snowwhite V28EP ** | 30% w/w |
| Mineral oil Carnation ** | 30% w/w |

* Available from Procter & Gamble Chemicals, Cincinnati, USA
** Available from Witco

The lotion composition has a melting point of about 51° C. and a melt viscosity at 56° C. of about 17 m*Pas measured at a shear rate of 0.1 l/s. The mineral oil used in this formulation has a viscosity of about 21 mpa*s at 20° C.

EXEMPLARY LOTION COMPOSITION #2 (nominal w/w)

| | |
|---|---|
| Mineral oil * | 55% w/w |
| Paraffin ** | 12% w/w |
| Cetaryl alcohol | 21% w/w |
| Steareth-2 *** | 11% w/w |
| Skin benefit agent | 1% w/w |

* Drakeol 7PG available from Penreco
** Chevron 128 available from Chevron
*** Available from Abitec Corporation

EXEMPLARY LOTION COMPOSITION #3 (nominal w/w)

| | |
|---|---|
| Mineral Oil* | 54.8% w/w |
| Cetearyl Alcohol | 28% w/w |
| Microcrystalline Wax | 12% w/w |
| Paraffin Wax** | 6% w/w |
| Aloe | 1.02% w/w |
| Vitamin E | 0.11% w/w |
| Shea Butter | 0.09% w/w |

*Drakeol 7PG available from Penreco
**Chevron 128 available from Chevron

The present invention contains as an essential component from about 2.0% to about 25.0% and preferably from 4.0% to about 11.0% of lotion based on the dry fiber weight of the tissue paper. In another embodiment, the present invention may contain as an essential component an application of from about 0.1 g/m² to about 30 g/m², preferably from about 0.55 g/m² to about 16.3 g/m², and more preferably from about 0.65 g/m² to about 10 g/m² of a lotion to the tissue paper.

### Facial Tissue

The facial tissue of the present invention further comprises papermaking fibers of both hardwood and softwood types wherein at least about 50% of the papermaking fibers are hardwood and at least about 10% are softwood. The hardwood and softwood fibers are most preferably isolated by relegating each to separate layers wherein the tissue comprises an inner layer and at least one outer layer.

The paper web which is first formed on a foraminous forming carrier, such as a Fourdrinier wire, where it is freed of the copious water needed to disperse the fibrous slurry is generally transferred to a felt or fabric in a so-called press section where de-watering is continued either by mechanically compacting the paper or by some other de-watering method such as through-drying with hot air, before finally being transferred in the semi-dry condition to the surface of the Yankee for the drying to be completed.

The facial tissue of the present invention is preferably creped, i.e., produced on a papermaking machine culminating with a Yankee dryer to which a partially dried papermaking web is adhered and upon which it is dried and from which it is removed by the action of a flexible creping blade.

Creping is a means of mechanically compacting paper in the machine direction. The result is an increase in basis weight (mass per unit area) as well as dramatic changes in many physical properties, particularly when measured in the machine direction. Creping is generally accomplished with a flexible blade, a so-called doctor blade, against a Yankee dryer in an on machine operation. A Yankee dryer is a large diameter, generally 8-20 foot drum which is designed to be pressurized with steam to provide a hot surface for completing the drying of papermaking webs at the end of the papermaking process.

While the characteristics of the creped paper webs, particularly when the creping process is preceded by methods of pattern densification, are preferred for practicing the present invention, un-creped tissue paper is also a satisfactory substitute and the practice of the present invention using un-creped tissue paper is specifically incorporated within the scope of the present invention. Un-creped tissue paper, a term as used herein, refers to tissue paper which is non-compressively dried, most preferably by through-drying. Resultant through air dried webs are pattern densified such that zones of relatively high density are dispersed within a high bulk field, including pattern densified tissue wherein zones of relatively high density are continuous and the high bulk field is discrete.

To produce un-creped tissue paper webs, an embryonic web is transferred from the foraminous forming carrier upon which it is laid, to a slower moving, high fiber support transfer fabric carrier. The web is then transferred to a drying fabric upon which it is dried to a final dryness. Such webs can offer some advantages in surface smoothness compared to creped paper webs.

Tissue paper webs are generally comprised essentially of papermaking fibers. Small amounts of chemical functional agents such as wet strength or dry strength binders, retention aids, surfactants, size, chemical softeners, crepe facilitating compositions are frequently included but these are typically only used in minor amounts. The papermaking fibers most frequently used in tissue papers are virgin chemical wood pulps. Filler materials may also be incorporated into the tissue papers of the present invention. Additionally, softening agents such as quaternary ammonium compounds can be added to the papermaking slurry. References disclosing softening agents such as polysiloxanes include U.S. Pat. Nos. 2,826,551; 3,964,500; 4,364,837; 5,059,282; 5,529,665; 5,552,020; and British Patent 849,433.

### Optional Chemical Additives

Other materials can be added to the aqueous papermaking furnish or the embryonic web to impart other characteristics to the product or improve the papermaking process so long as they are compatible with the chemistry of the softening agent and do not significantly and adversely affect the softness, strength, or low dusting character of the present invention. The following materials are expressly included, but their inclusion is not offered to be all-inclusive. Other materials can be included as well so long as they do not interfere or counteract the advantages of the present invention. This can include polyamide-epichlorohydrin resins (cationic wet strength resins) which have been found to be of particular utility. Suitable types of such resins are described in U.S. Pat. Nos. 3,700,623 and 3,772,076.

The present invention is further applicable to the production of multi-layered tissue paper webs. Multi-layered tissue structures and methods of forming multi-layered tissue structures are described in U.S. Pat. Nos. 3,994,771; 4,300,981; 4,166,001; and European Patent Publication No. 0 613 979 A1. The tissue paper products made from single-layered or multi-layered un-creped tissue paper webs can be single-ply tissue products or multi-ply tissue products.

The multi-layered tissue paper webs of to the present invention can be used in any application where soft, absorbent multi-layered tissue paper webs are required. Particularly advantageous uses of the multi-layered tissue paper web of this invention are in toilet tissue and facial tissue products. Both single-ply and multi-ply tissue paper products can be produced from the webs of the present invention.

### Application of a Lotion to Paper Webs

In accordance with the present invention, the lotion compositions may be applied to a paper web by any application method known in the industry such as, for example, spraying, printing, extrusion, brushing, by means of permeable or impermeable rolls and/or pads. In a first embodiment, the lotion compositions may be applied to a paper web with a slot die. Specifically, the lotion compositions may be extruded onto the surface of a paper web via a heated slot die. The slot die may be any suitable slot die or other means for applying a lotion compositions to the paper web. The slot die means may be supplied by any suitable apparatus. For example, the slot die may be supplied by a heated hopper or drum and a variable speed gear pump through a heated hose. The lotion composition is preferably extruded onto the surface of the paper web at a temperature that permits the lotion composition to bond to the paper web. Depending on the particular embodiment, the lotion composition can be at least partially transferred to rolls in a metering stack (if used) and then to the paper web.

Additionally, the lotion composition may be applied to a paper web by an apparatus comprising a fluid transfer component. The fluid transfer component preferably comprises a first surface and a second surface. The fluid transfer component further preferably comprises pores connecting the first surface and the second surface. The pores are disposed upon the fluid transfer component in a non-random pre-selected pattern. A fluid supply is operably connected to the fluid transfer component such that a fluid (such as the lotion composition) may contact the first surface of the fluid transfer component. The apparatus further comprises a fluid motivating component. The fluid motivating component provides an impetus for the fluid to move from the first surface to the second surface via the pores. The apparatus further comprises a fluid receiving component comprising a paper web. The paper web comprises a fluid receiving (or outer) surface. The fluid receiving surface may contact droplets of fluid formed upon the second surface. Fluid may pass through pores from the first surface to the second surface and may transfer to the fluid receiving surface.

The fluid transfer component may comprise a hollow cylindrical shell. The cylindrical shell may be sufficiently structural to function without additional internal bracing. The cylindrical shell may comprise a thin outer shell and structural internal bracing to support the cylindrical shell. The cylindrical shell may comprise a single layer of material or may comprise a laminate. The laminate may comprise layers of a similar material or may comprise layers dissimilar in material and structure. In one embodiment the cylindrical shell comprises a stainless steel shell having a wall thickness of about 0.125 inches (3 mm). In another embodiment the fluid transfer component may comprise a flat plate. In another embodiment the fluid transfer component may comprise a regular or irregular polygonal prism.

The fluid application width of the apparatus may be adjusted by providing a single fluid transfer component of appropriate width. Multiple individual fluid application components may be combined in a series to achieve the desired width. In a non-limiting example, a plurality of stainless steel cylinders each having a shell thickness of about 0.125 inches (3 mm) and a width of about 6 inches (about 15 cm) may be coupled end to end with an appropriate seal—such as an o-ring seal between each pair of cylinders. In this example, the number of shells combined may be increased until the desired application width is achieved.

The fluid transfer component preferably further comprises pores connecting the first surface and the second surface. Connecting the surfaces refers to the pores each providing a pathway for the transport of a fluid from the first surface to the second surface. In one embodiment, the pores may be formed by the use of electron beam drilling as is known in the art. Electron beam drilling comprises a process whereby high energy electrons impinge upon a surface resulting in the formation of holes through the material. In another embodiment, the pores may be formed using a laser. In another embodiment, the pores may be formed by using a drill bit. In yet another embodiment, the pores may be formed using electrical discharge machining as if known in the art.

In one embodiment, an array of pores may be disposed to provide a uniform distribution of fluid droplets to maximize the ratio of fluid surface area to applied fluid volume. In one embodiment, this may be used to apply a lotion composition in a pattern of dots to maximize the potential for adhesion between two surfaces for any volume of applied chemical softening agent.

The pattern of pores upon the second surface may comprise an array of pores having a substantially similar diameter or may comprise a pattern of pores having distinctly different pore diameters. In an alternative embodiment, the array of pores may comprise a first set of pores having a first diameter and arranged in a first pattern. The array further comprises a second set of pores having a second diameter and arranged in a second pattern. The first and second patterns may be arranged to interact each with the other.

Alternatively, the lotion composition may be sprayed directly onto the surface of a paper web using equipment suitable for such a purpose and as well known to those of skill in the art.

### Example 1

A 3% by weight aqueous slurry of NSK (northern softwood Kraft) is made in a conventional re-pulper. The NSK slurry is refined, and a 2% solution of Kymene 557LX is added to the NSK stock pipe at a rate sufficient to deliver 1% Kymene 557LX by weight of the dry fibers. The absorption of the wet strength resin is enhanced by passing the treated slurry though an in-line mixer. KYMENE 557LX is supplied by Hercules Corp of Wilmington, Del. A 1% solution of carboxy methyl cellulose is added after the in-line mixer at a rate of 0.15% by weight of the dry fibers to enhance the dry strength of the fibrous structure. The aqueous slurry of NSK fibers passes through a centrifugal stock pump to aid in distributing the CMC. An aqueous dispersion of DiTallow DiMethyl Ammonium Methyl Sulfate (DTDMAMS) (170° F./76.6° C.) at a concentration of 1% by weight is added to the NSK stock pipe at a rate of about 0.05% by weight DTDMAMS per ton of dry fiber weight.

A 3% by weight aqueous slurry of eucalyptus fibers is made in a conventional re-pulper. A 2% solution of Kymene 557LX is added to the eucalyptus stock pipe at a rate sufficient to deliver 0.25% Kymene 557LX by weight of the dry fibers. The absorption of the wet strength resin is enhanced by passing the treated slurry though an in-line mixer.

The NSK fibers are diluted with white water at the inlet of a fan pump to a consistency of about 0.15% based on the total weight of the NSK fiber slurry. The eucalyptus fibers, likewise, are diluted with white water at the inlet of a fan pump to a consistency of about 0.15% based on the total weight of the eucalyptus fiber slurry. The eucalyptus slurry and the NSK slurry are directed to a multi-channeled headbox suitably equipped with layering leaves to maintain the streams as separate layers until discharged onto a traveling Fourdrinier wire. A three-chambered headbox is used. The eucalyptus slurry containing 65% of the dry weight of the tissue ply is directed to the chamber leading to the layer in contact with the wire, while the NSK slurry comprising 35% of the dry weight of the ultimate tissue ply is directed to the chamber leading to the center and inside layer. The NSK and eucalyptus slurries are combined at the discharge of the headbox into a composite slurry.

The composite slurry is discharged onto the traveling Fourdrinier wire and is dewatered assisted by a deflector and vacuum boxes. The Fourdrinier wire is of a 5-shed, satin weave configuration having 105 machine-direction and 107 cross-machine-direction monofilaments per inch. The speed of the Fourdrinier wire is about 800 fpm (feet per minute).

The embryonic wet web is dewatered to a consistency of about 15% just prior to transfer to a patterned drying fabric made in accordance with U.S. Pat. No. 4,529,480. The speed of the patterned drying fabric is the same as the speed of the Fourdrinier wire. The drying fabric is designed to yield a pattern-densified tissue with discontinuous low-density deflected areas arranged within a continuous network of high density (knuckle) areas. This drying fabric is formed by casting an impervious resin surface onto a fiber mesh supporting fabric. The supporting fabric is a 45×52 filament, dual layer mesh. The thickness of the resin cast is about 0.009 inches above the supporting fabric. The drying fabric for forming the paper web has about 562 discrete deflection regions per square inch. The area of the continuous network is about 50 percent of the surface area of the drying fabric.

Further dewatering is accomplished by vacuum assisted drainage until the web has a fiber consistency of about 25%. While remaining in contact with the patterned drying fabric, the web is pre-dried by air blow-through pre-dryers to a fiber consistency of about 65% by weight. The web is then adhered to the surface of a Yankee dryer, and removed from the surface of the dryer by a doctor blade at a consistency of about 97 percent. The Yankee dryer is operated at a surface speed of about 800 feet per minute. The dry web is passed through a rubber-on-steel calendar nip. The dry web is wound onto a roll at a speed of 680 feet per minute to provide dry foreshortening of about 15 percent. The resulting web has between about 562 and about 650 relatively low density domes per square inch (the number of domes in the web is between zero percent to about 15 percent greater than the number of cells in the drying fabric, due to dry foreshortening of the web).

Two plies were combined with the wire side facing out. During the converting process, a surface softening agent and a lotion (exemplary lotion composition #2 described supra) are applied sequentially with slot extrusion dies to the outside surface of both plies. The surface softening agent is a formula comprising one or more polyhydroxy compounds (Polyethylene glycol, Polypropylene glycol, and/or copolymers thereof marketed by BASF Corporation of Florham Park, N.J.), glycerin (marketed by PG Chemical Company), and silicone (i.e. MR-1003, marketed by Wacker Chemical Corporation of Adrian, Mich.). The surface softening agent is applied to the web at a rate of 14.1% by weight and the lotion is applied to the web at a rate of 5.0% by weight. The plies are then bonded together with mechanical ply-bonding wheels, slit, and then folded into finished 2-ply facial tissue product. Ten 2-ply folded facial tissue products are then assembled, stacked, and packaged in a film material (discussed infra) to form package of 10 folded and lotioned facial tissues. Individual folded facial tissues and packages of 10 folded, stacked, and packaged facial tissues are tested in accordance with the test methods described infra.

### Example 2

A web is produced according to the process described in Example 1. Two plies were combined with the wire side facing out. During the converting process, a surface softening agent and a lotion (i.e., exemplary lotion composition #3 described supra) are applied sequentially with slot extrusion dies to the outside surface of both plies. The surface softening agent is a formula comprising one or more polyhydroxy compounds (Polyethylene glycol, Polypropylene glycol, and/or copolymers thereof marketed by BASF Corporation of Florham Park, N.J.), glycerin (marketed by PG Chemical Company), and silicone (i.e. MR-1003, marketed by Wacker Chemical Corporation of Adrian, Mich.). The surface softening agent is applied to the web at a rate of 14.1% by weight and the lotion is applied to the web at a rate of 5.0% by weight. The plies are then bonded together with mechanical ply-bonding wheels, slit, and then folded into finished 2-ply facial tissue product. Ten 2-ply folded facial tissue products are then assembled, stacked, and packaged in a film material (discussed infra) to form package of 10 folded and lotioned facial tissues. Individual folded facial tissues and packages of 10 folded, stacked, and packaged facial tissues are tested in accordance with the test methods described infra.

### Example 3

A web is produced according to the process described in Example 1. Two plies were combined with the wire side facing out. During the converting process, a surface softening agent and a lotion (i.e., exemplary lotion composition #3 described supra) are applied sequentially with slot extrusion dies to the outside surface of both plies. The surface softening agent is a formula comprising one or more polyhydroxy compounds (Polyethylene glycol, Polypropylene glycol, and/or copolymers thereof marketed by BASF Corporation of Florham Park, N.J.), glycerin (marketed by PG Chemical Company), and silicone (i.e. MR-1003, marketed by Wacker Chemical Corporation of Adrian, Mich.). The surface softening agent is applied to the web at a rate of 14.1% by weight and the lotion is applied to the web at a rate of 10.0% by weight. The plies are then bonded together with mechanical ply-bonding wheels, slit, and then folded into finished 2-ply facial tissue product. Ten 2-ply folded facial tissue products are then assembled, stacked, and packaged in a film material (discussed infra) to form package of 10 folded and lotioned facial tissues. Individual folded facial tissues and packages of 10 folded, stacked, and packaged facial tissues are tested in accordance with the test methods described infra.

### Packaging

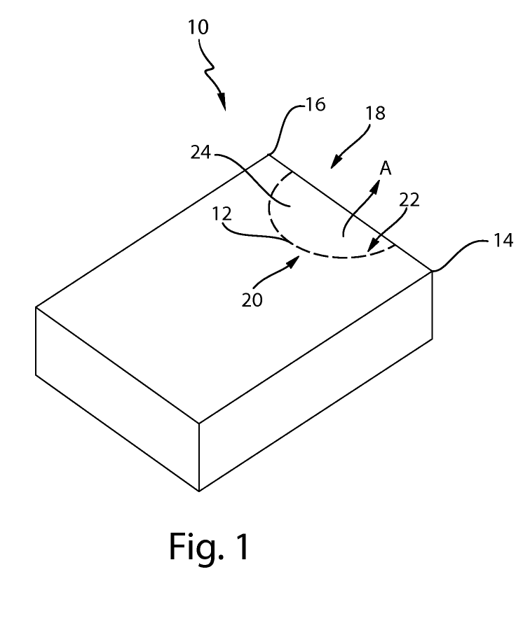

FIG. 1 shows a perspective view of an exemplary facial tissue package 10 having a resealable opening. The package material is preferably a thin flexible plastic film that has been folded and sealed around a small stack of folded tissues. The opening 22 is preferably semi-circular and is partially defined by perforations 12 in the plastic film which emanate from the top panel 18 in a region proximate to the corners 14 and 16 of the package 10.

In use, the user grasps the distal end 20 of the opening flap 24 and pulls in the direction of the arrow A to break the perforations 12 and pull back the opening flap 24, thereby exposing the tissues inside. The shape of the opening flap 24 is not critical, although the size of the opening in the package 10 must be large enough to allow removal of the tissues without tearing them, yet small enough to contain the tissues within the pack when the flap is open.

As illustrated in FIG. 1, the distance between the terminal portions of the opening flap 24 formed by perforations 12 is generally about 1.75 inches. An opening flap 24 having a semi-circular shape is preferred because a semi-circular opening flap 24 can eliminate or substantially eliminate any exposed corners which might otherwise detract from the appearance of the package 10 after the package 10 has been in use for some time. The perforations 12 can even wrap around the edges of the package 10 provided the opening is of a size which functions properly. The perforations 12 can also follow any curvilinear or straight line to form a wide variety of opening flap 24 and dispensing opening shapes.

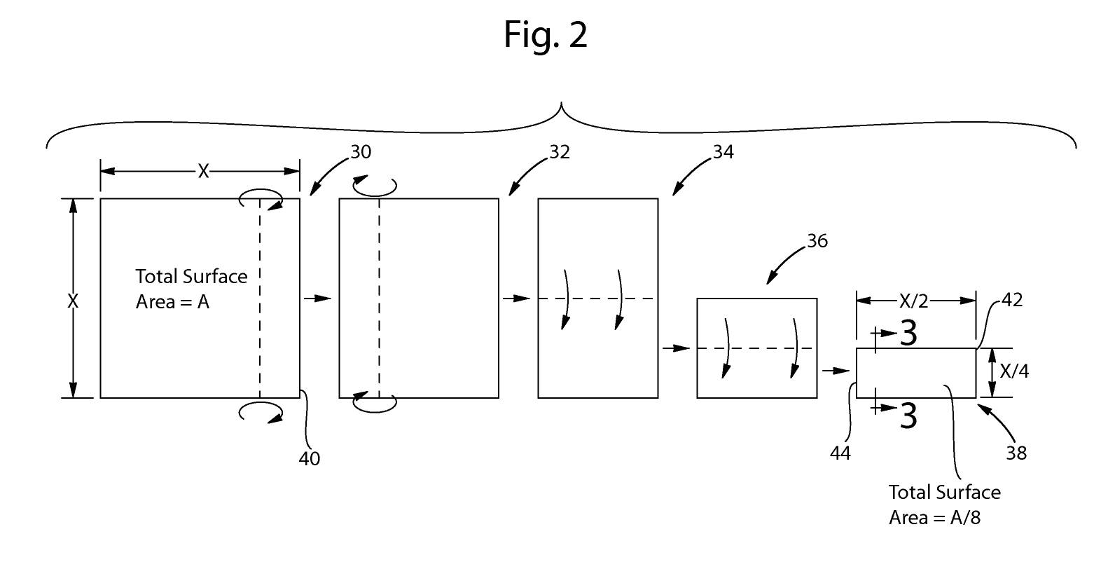

FIG. 2 shows an exemplary and clearly non-limiting process for folding a facial tissue 30 of the present disclosure having nominal dimensions of 8 inches by 8 inches for insertion into a facial tissue package 10. For purposes of the present disclosure the folded facial tissue 30 is generally provided in a form of a parallelogram where each side is provided with a dimension of X. The folded configuration illustrated in the second figure of the folding sequence is referred to as a “z-folded” configuration 32, in which opposite edges of the tissue are folded to place both edges at or proximate to the centerline of the tissue sheet 30.

The dashed lines indicate the fold lines used to produce a finally folded tissue sheet 38. After the z-folded tissue 32 is completed, the z-folded tissue 32 is folded again to fold the z-folded tissue 32 in half to give the configuration 36 shown in the third figure of the sequence. Then, as before, the tissue is again folded in half where indicated by the dashed line to give the finally folded tissue sheet 38. As shown, the edge 40 of the tissue sheet 30 is exposed on the face of the finally folded tissue sheet 38. Generally, the edge 40 can be disposed midway between and parallel to opposite sides 42 and 44 of the finally folded tissue sheet 38. The finally folded tissue sheet 38 of the present disclosure will have nominal dimensions of 4″×2″ based upon an original unfolded facial tissue having nominal dimensions of 8″×8″. It is interesting to note that if the unfolded tissue 30 has an unfolded surface area of A the finally folded tissue sheet 38 will have a surface area of A/8. In other words, the finally folded tissue sheet 38 will have a longitudinal axis of X/2 and a dimension of X/4 for the coplanar axis orthogonal thereto. However, one of skill in the art will clearly understand that the tissue sheet 30 can be provided with any dimensions as may be required by the producer and consumer of the finally folded product and may have a finally folded configuration and/or dimensions that may be required by the producer and consumer of the finally folded product.

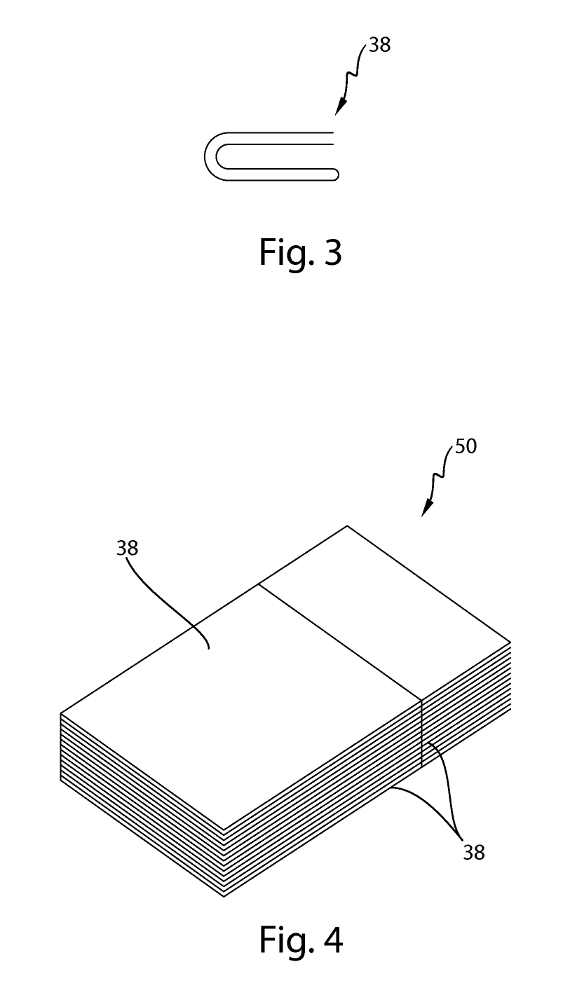

FIG. 3 is a cross sectional view of the finally folded tissue sheet 38 of FIG. 2. As can be seen finally folded tissue sheet 38 has a cross-sectional geometry that would likely be referred to by one of skill in the art as a double “V”-fold.

FIG. 4 is a perspective view of a stack 50 of individually finally folded tissue sheets 38. All of the tissues in the stack 50 are preferably oriented the same as the finally folded tissue sheet 38 on the top of the stack 50. However, one of skill in the art would recognize that the finally folded tissue sheets 38 comprising stack 50 could have any orientation required to accomplish the goals of dispensing required by the user.

### Analytical and Testing Procedures

The following test methods are representative of the techniques utilized to determine the physical characteristics of the multi-ply tissue product associated therewith.

### 1. Sample Conditioning and Preparation

Unless otherwise indicated, samples are conditioned according to Tappi Method #T402OM-88. Samples are conditioned for at least 2 hours at a relative humidity of 48 to 52% and within a temperature range of 22° to 24° C. Sample preparation and all aspects of testing using the following methods are confined to a constant temperature and humidity room.

### 2. Basis Weight

Basis weight is measured by preparing one or more samples of a certain area (m²) and weighing the sample(s) of a fibrous structure according to the present invention and/or a paper product comprising such fibrous structure on a top loading balance with a minimum resolution of 0.01 g. The balance is protected from air drafts and other disturbances using a draft shield.

Weights are recorded when the readings on the balance become constant. The average weight (g) is calculated and the average area of the samples (m²). The basis weight (g/m²) is calculated by dividing the average weight (g) by the average area of the samples (m²).

The facial tissue of the present disclosure preferably has a basis weight ranging from between about 5 g/m² and about 120 Wm², more preferably between about 10 g/m² and about 75 g/m², and even more preferably between about 10 g/m² and about 50 g/m². The facial tissue of the present invention preferably has a density ranging from between about 0.01 g/cm³ and about 0.19 g/cm³, more preferably between about 0.02 g/m³ and about 0.1 g/cm³, and even more preferably between about 0.03 g/cm³ and about 0.08 g/cm³.

### 3. Density

The density of a facial tissue is the average density calculated as the basis weight of that paper divided by the caliper, with the appropriate unit conversions incorporated therein. Caliper of the multi-layered tissue paper is the thickness of the paper when subjected to a compressive load of 95 g/in² (14.7 g/cm²).

### 4. Caliper Test Method

Caliper of a fibrous structure or package is measured by providing five (5) samples of fibrous structure so that each cut sample is larger in size than a load foot loading surface of a VIR Electronic Thickness Tester Model II available from Thwing-Albert Instrument Company, Philadelphia, Pa. Typically, the load foot loading surface has a circular surface area of about 3.14 in². The sample is confined between a horizontal flat surface and the load foot loading surface. The load foot loading surface applies a confining pressure to the sample of 15.5 g/cm².

Each tissue sample to be tested is folded to provide a 4-ply structure. For example only, a 2-ply tissue sample is “V”-folded folded once to provide a 4-ply sample for testing having the surface area stated supra. For example only, a 1-ply tissue sample is “V”-folded twice to provide a 4-ply sample for testing having the surface area stated supra. The caliper of each sample is the resulting gap between the flat surface and the load foot loading surface. The caliper is calculated as the average caliper of the five samples. The result is reported in millimeters (mm).

Each product sample to be tested the packaged product is placed within the tester as provided above. The caliper of each product sample is the resulting gap between the flat surface and the load foot loading surface. The caliper is calculated as the average caliper of the five samples. The result is reported in millimeters (mm).

### 5. Wet Burst

For the purposes of determining, calculating, and reporting ‘wet burst’, ‘total dry tensile’, and ‘dynamic coefficient of friction’ values infra, a unit of ‘user units’ is hereby utilized for the products subject to the respective test method. As would be known to those of skill in the art, bath tissue and paper toweling are typically provided in a perforated roll format where the perforations are capable of separating the tissue or towel product into individual units. A ‘user unit’ (uu) is the typical finished product unit that a consumer would utilize in the normal course of use of that product. A single-, double, or even triple-ply finished product that a consumer would normally use would have a value of one user unit (uu). For example, facial tissues that are not normally provided in a roll format, but as a stacked plurality of discreet tissues, a facial tissue having one ply would have a value of 1 user unit (uu). An individual two-ply facial tissue product would have a value of one user unit (1 uu), etc.

Wet burst strength is measured using a Thwing-Albert Intelect II STD Burst Tester. 8 uu of tissue are stacked in four groups of 2 uu. Using scissors, cut the samples so that they are approximately 208 mm in the machine direction and approximately 114 mm in the cross-machine direction, each 2 uu thick.

Take one sample strip, holding the sample by the narrow cross direction edges, dipping the center of the sample into a pan filled with about 25 ml of distilled water. Leave the sample in the water four (4.0+/−0.5) seconds. Remove and drain for three (3.0+/−0.5) seconds holding the sample so the water runs off in the cross direction. Proceed with the test immediately after the drain step. Place the wet sample on the lower ring of the sample holding device with the outer surface of the product facing up, so that the wet part of the sample completely covers the open surface of the sample holding ring. If wrinkles are present, discard the sample and repeat with a new sample. After the sample is properly in place on the lower ring, turn the switch that lowers the upper ring. The sample to be tested is now securely gripped in the sample holding unit. Start the burst test immediately at this point by pressing the start button. The plunger will begin to rise. At the point when the sample tears or ruptures, report the maximum reading. The plunger will automatically reverse and return to its original starting position. Repeat this procedure on three more samples for a total of four tests, i.e., 4 replicates. Average the four replicates and divide this average by two to report wet burst per uu, to the nearest gram.

### 6. Total Dry Tensile Strength

The tensile strength is determined on one inch wide strips of sample using a Thwing Albert Vontage-10 Tensile Tester (Thwing-Albert Instrument Co., 10960 Dutton Rd., Philadelphia, Pa., 19154). This method is intended for use on finished paper products, reel samples, and unconverted stocks.

a. Sample Conditioning and Preparation

Prior to tensile testing, the paper samples to be tested are conditioned according to Tappi Method #T402OM-88. The paper samples should be conditioned for at least 2 hours at a relative humidity of 48% to 52% and within a temperature range of 22° to 24° C. Sample preparation and all aspects of the tensile testing should also take place within the confines of the constant temperature and humidity room.

For finished products, discard any damaged product. Take 8 uu of tissue and stack them in four stacks of 2 uu. Use stacks 1 and 3 for machine direction tensile measurements and stacks 2 and 4 for cross direction tensile measurements. Cut two f-inch wide strips in the machine direction from stacks 1 and 3. Cut two 1-inch wide strips in the cross direction from stacks 2 and 4. There are now four 1″ wide strips for machine direction tensile testing and four 1-inch wide strips for cross direction tensile testing. For these finished product samples, all eight 1″ wide strips are 2 uu thick.

For unconverted stock and/or reel samples, cut a 15-inch by 15-inch sample which is twice the number of plies in a user unit thick from a region of interest of the sample using a paper cutter (JDC-1-10 or JDC-1-12 with safety shield from Thwing-Albert Instrument Co., 10960 Dutton Road, Philadelphia, Pa. 19154). Make sure one 15-inch cut runs parallel to the machine direction while the other runs parallel to the cross direction. Make sure the sample is conditioned for at least 2 hours at a relative humidity of 48% to 52% and within a temperature range of 22° C. to 24° C. Sample preparation and all aspects of the tensile testing should also take place within the confines of the constant temperature and humidity room.

From this preconditioned 15-inch by 15-inch sample which is twice the number of plies in a user unit thick, cut four strips 1-inch by 7-inch with the long 7-inch dimension running parallel to the machine direction. Note these samples as machine direction reel or unconverted stock samples. Cut an additional four strips 1-inch by 7-inch with the long 7-inch dimension running parallel to the cross direction. Note these samples as cross direction reel or unconverted stock samples. Make sure all previous cuts are made using a paper cutter (JDC-1-10 or JDC-1-12 with safety shield from Thwing-Albert Instrument Co., 10960 Dutton Road, Philadelphia, Pa., 19154). There are now a total of eight samples: four 1-inch by 7-inch strips which are twice the number of plies in a uu thick with the 7-inch dimension running parallel to the machine direction and four 1-inch by 7-inch strips which are twice the number of plies in a uu thick with the 7-inch dimension running parallel to the cross direction.

b. Operation of Tensile Tester

For the actual measurement of the tensile strength, use a Thwing Albert Vontage-10 Tensile Tester (Thwing-Albert Instrument Co., 10960 Dutton Rd., Philadelphia, Pa., 19154). Insert the flat face clamps into the unit and calibrate the tester according to the instructions given in the operation manual of the Thwing Albert Vontage-10. Set the instrument crosshead speed to 2.00 in/min and the 1st and 2nd gauge lengths to 4.00 inches. The break sensitivity should be set to 20.0 grams and the sample width should be set to 1.00 inches and the sample thickness at 0.025 inches.

A load cell is selected such that the predicted tensile result for the sample to be tested lies between 25% and 75% of the range in use. For example, a 5000 gram load cell may be used for samples with a predicted tensile range of 1250 grams (25% of 5000 grams) and 3750 grams (75% of 5000 grams). The tensile tester can also be set up in the 10% range with the 5000 gram load cell such that samples with predicted tensile strengths of 125 grams to 375 grams could be tested.

Take one of the tensile strips and place one end of it in one clamp of the tensile tester. Place the other end of the paper strip in the other clamp. Make sure the long dimension of the strip is running parallel to the sides of the tensile tester. Also make sure the strips are not overhanging to the either side of the two clamps. In addition, the pressure of each of the clamps must be in full contact with the paper sample.

After inserting the paper test strip into the two clamps, the instrument tension can be monitored. If it shows a value of 5 grams or more, the sample is too taut. Conversely, if a period of 2-3 seconds passes after starting the test before any value is recorded, the tensile strip is too slack.

Start the tensile tester as described in the tensile tester instrument manual. The test is complete after the crosshead automatically returns to its initial starting position. Read and record the tensile load in units of grams from the instrument scale or the digital panel meter to the nearest unit.

If the reset condition is not performed automatically by the instrument, perform the necessary adjustment to set the instrument clamps to their initial starting positions. Insert the next paper strip into the two clamps as described above and obtain a tensile reading in units of grams. Obtain tensile readings from all the paper test strips. It should be noted that readings should be rejected if the strip slips or breaks in or at the edge of the clamps while performing the test.

c. Calculations

For the four machine direction 1-inch wide finished product strips, average the four individual recorded tensile readings. Divide this average by the number of user unit tested to get the MD dry tensile per user unit of the sample. Repeat this calculation for the cross direction finished product strips. To calculate total dry tensile of the sample, sum the MD dry tensile and CD dry tensile. All results are in units of grams/inch.

To calculate the Wet Burst/Total Dry Tensile ratio divide the average wet burst by the total dry tensile. The results are in units of inches.

### 7. Dynamic Coefficient of Friction

The dynamic coefficient of friction is measured using a Thwing-Albert Friction/Peel Tester Model 225-1. The Friction test is set up by pressing the C.O.F button on the Display Unit to select the Friction Test. The Friction Tester operated with a 2000 gram Load Cell, a padded cell of 200 grams at a speed of 6 in/min over 20 seconds. The test is initiated by depressing the Test Switch on the lower chassis of the front panel. The Load Cell will travel to the right, pulling the sled along with the affixed sample. The test results are displayed on an LCD panel. The display indicates the force in grams required for the sled to move along the test surface, i.e. the friction between usable units along with the static and dynamic coefficients of friction (COF). The displayed force returns to zero after the sled is removed from the test surface.

Ten usable units of tissue are stacked in two sets of five. Using scissors, cut one set of 5 usable units so that they are approximately 153 mm in the machine direction and approximately 114 mm in the cross-machine direction. Do not alter the second set of five usable units.

Using the test surface clamp and double sided tape, take one of the five unaltered usable units and affix to the test surface of the machine. Then, affix one usable unit of the five prepared 153 mm×114 mm prepared samples to the sled. Connect the sled to the Load Cell via the sled hook. Ensure that the LCD load (LD) reads 0.0 grams, that the sample is centered, and that the connecting wire is taut. Initiate the test by depressing the Test Switch on the lower chassis of the front panel. The results will display on the LCD panel. Remove the sled along with the usable unit from the test surface. Remove the 153 mm×114 mm usable unit from the sled. Load new usable units to the test surface and 153 mm×114 mm usable unit to the sled. Return the Load Cell to the starting position for the next test. Repeat test procedure 4 times. The five data points collected for COF are recorded and averaged for each sample condition.

### 8. Bending Flexibility

a. Equipment:

Tissue flexibility is measured using the Kawabata KES-FB2 Pure Bending Tester instrument (KES Kato Tech Co., LTD., 26 Karato-cho Nishikujo Minami-ku, Kyoto 601 Japan) to measure flexural rigidity by bending a sample at a constant rate of curvature change in two directions while measuring the bending moment. The sample is held between two clamps 1 cm apart. The typical tissue sample width used is approximately 10-21 cm. Curvature, K, is the reciprocal of the radius of the bending circle. The sample is bent at a constant rate of curvature change of 0.5 cm⁻¹/sec, starting at K=0, to K=2.35 (±0.03) back to K=0, then to K=−2.5 (±0.03) then finally back to K=0 (K in units cm⁻¹). As the sample is bent, force is measured on a stationary grip. The data results of the full cycle of bending are bending moment (per unit sample width) versus curvature (cm⁻¹). The data from each test is saved as a file for subsequent analysis.

b. Method for Measuring Flexibility of a Non-Lotioned Tissue:

Tissue product samples are cut to approximately 15.2 cm×20.3 cm in the machine and cross machine directions, respectively. Each sample in turn is placed in the jaws of the KES-FB2 such that the sample would first be bent with the first surface undergoing tension and the second surface undergoing compression. In the orientation of the KES-FB2 the first surface is right facing and the second surface is left facing. The distance between the front moving jaw and the rear stationary jaw is 1 cm. The sample is secured in the instrument in the following manner.

First the front moving chuck and the rear stationary chuck are opened to accept the sample. The sample is inserted midway between the top and bottom of the jaws. The rear stationary chuck is then closed by uniformly tightening the upper and lower thumb screws until the sample is snug, but not overly tight. The jaws on the front stationary chuck are then closed in a similar fashion. The sample is adjusted for squareness in the chuck, then the front jaws are tightened to insure the sample is held securely. The distance (d) between the front chuck and the rear chuck is 1 cm.

The output of the instrument is load cell voltage (Vy) and curvature voltage (Vx). The load cell voltage is converted to a bending moment (M) normalized for sample width in the following manner:

Moment (M, gf*cm²/cm)=(Vy*Sy*d)/W
Where:
   Vy is the load cell voltage,
   Sy is the instrument sensitivity in gf*cmN,
   d is the distance between the chucks, and
   W is the sample width in centimeters.

The sensitivity switch of the instrument is set at 5×1. Using this setting the instrument is calibrated using two 50 g weights. Each weight is suspended from a thread. The thread is wrapped around the bar on the bottom end of the rear stationary chuck and hooked to a pin extending from the front and back of the center of the shaft. One weight thread is wrapped around the front and hooked to the back pin. The other weight thread is wrapped around the back of the shaft and hooked to the front pin. Two pulleys are secured to the instrument on the right and left side. The top of the pulleys are horizontal to the center pin. Both weights are then hung over the pulleys (one on the left and one on the right) at the same time. The full scale voltage is set at 10 V. The radius of the center shaft is 0.5 cm. Thus the resultant full scale sensitivity (Sy) for the Moment axis is 100 gf*0.5 cm/10V (5 gf*cmN).

The output for the Curvature axis is calibrated by starting the measurement motor and manually stopping the moving chuck when the indicator dial reached 1.0 cm⁻¹. The output voltage (Vx) is adjusted to 0.5 volts. The resultant sensitivity (Sx) for the curvature axis is 2/(volts*cm). The curvature (K) is obtained in the following manner:

Curvature (K, cm−1)=Sx*Vx
Where:
Sx is the sensitivity of the curvature axis, and
Vx is the output voltage

For determination of the bending stiffness the moving chuck is cycled from a curvature of 0 cm⁻¹ to +1 cm⁻¹ to −1 cm⁻¹ to 0 cm⁻¹ at a rate of 0.5 cm−1/sec. Each sample is cycled continuously until four complete cycles are obtained. The output voltage of the instrument is recorded in a digital format using a personal computer. A typical output for a bending stiffness test is shown in FIG. 4. At the start of the test there is no tension on the sample. As the test begins the load cell begins to experience a load as the sample is bent. The initial rotation is clockwise when viewed from the top down on the instrument.

In the forward bend the first surface of the fabric is described as being in tension and the second surface is being compressed. The load continued to increase until the bending curvature reached approximately +1 cm⁻¹ (this is the Forward Bend (FB). At approximately +1 cm⁻¹ the direction of rotation is reversed. During the return the load cell reading decreases. This is the Forward Bend Return (FR). As the rotating chuck passes 0 curvature begins in the opposite direction—that is, the sheet side now compresses and the no-sheet side extends. The Backward Bend (BB) extended to approximately −1 cm⁻¹ at which the direction of rotation is reversed and the Backward Bend Return (BR) is obtained.

The data are analyzed in the following manner. A linear regression line is obtained between approximately 0.2 and 0.7 cm⁻¹ for the Forward Bend (FB) and the Forward Bend Return (FR). A linear regression line is obtained between approximately −0.2 and −0.7 cm⁻¹ for the Backward Bend (BB) and the Backward Bend Return (BR). The slope of the line is the Bending Stiffness (B). It has units of gf*cm²/cm.

This is obtained for each of the four cycles for each of the four segments. The slope of each line is reported as the Bending Stiffness (B). It has units of gf*cm²/cm. The Bending Stiffness of the Forward Bend is noted as BFB. The individual segment values for the four cycles are averaged and reported as an average BFB, BFR, BBF, BBR. Two separate samples in the MD and the CD are run. Values for the two samples are averaged together using the square root of the sum of the squares.

c. Method for Measuring Flexibility of a lotioned tissue:
1. Set-up and Calibration

Hardware: Turn measurement SENS (sensitivity) knob on equipment to 20. Turn the CHECK instrument knob to OSC—the needle gauge (voltmeter) on the instrument should equal 10+/−0.1 unit. Turn CHECK knob to BAL—the needle gauge on instrument should equal 0+/−0.1 unit. Adjust the AC BAL screw to move the needle into the acceptable range. Turn CHECK knob to ZERO—the gauge should equal 0+/−0.1 unit on the needle gauge. If not, use small screwdriver to turn the ZERO ADJ adjustment screw (front of instrument) to zero. Using a 20 gram weight connected to a fine silk thread with a loop on the end (such as is sold by Kato Tech Co. LTD) remove the back panel of the instrument and hang the 20 g weight from the pin extending from the stationary grip (also referred to as fixed chuck). The needle gauge should equal 10 units (±0.25 units). Connect a digital volt meter to the output terminals “T” and “E” on the instrument face. Record the voltage reading, then remove the 20 g weight from the stationary grip, and record the new voltage reading. The difference between the two voltage readings should with the acceptable range of 9.75 and 10.25 volts. If not, adjust the GAIN adjustment screw (with a flathead screwdriver) until the difference is within the acceptable range. Repeat this procedure until the difference in voltage (with and without 20 g weight attached) is within the acceptable range, then verify the OSC, BAL, and ZERO are in the acceptable range, as described earlier. When finished, turn the CHECK knob to MES—this is the measurement mode for the instrument.

Software: Change the SENS to read 2×1 (this correctly matches the software to the hardware sensitivity settings). Adjust the “Size” to read 20 cm, and the “Mode” to read one cycle. Settings for B and 2HB do not matter, since the raw data file from each test is analyzed separately from the software provided from Kato Tech Co.

2. Sample Preparation
Cut 5 tissue sample uu to approximately 20 cm (±1 cm) long in the machine direction (MD) by 15 cm (±1 cm) in the cross machine direction (CD). Folds that are present in the cut sample, created by the converting process used in making the uu, may be included in the measured test sample; however, any ply-seal marks near the sample edges (which may or may not include glue) are removed from the test sample and any effect upon the flexural rigidity measurement is excluded.

3. Measurement

Ensure that the CHECK knob is on MES. To test the MD of the first sample, lay one pre-cut uu sample on the flat chrome instrument sample plate, with the MD pointing towards to and from the person facing instrument front panel (the CD of the sample should be directed left and right relative to the user). Measure the sample width (CD direction) to the nearest 0.1 cm, at a distance approximately 1½ to 2½ inches from the sample end that will be fed into the instrument jaws (i.e., the end furthest from the person standing in front of the instrument). Record the distance (with respect to the sample ID) for later use in data analysis and calculations. Place the sample into the both jaws of the instrument, centered relative to the jaw width. When the sample is adequately positioned through both jaws, a small red light on the instrument illuminates to inform the tester that the test can begin (also, the MEASURE button will not function unless this occurs). Press the MEASURE button—this will cause the instrument to automatically close the jaws, clamping the sample into place. Once the MEASURE button begins to blink on and off, then, using the KES software program, provide a test name and start the measurement. The instrument bends the sample (at a rate of 0.5 cm⁻¹/sec) up to a curvature of K=2.35 (±0.03) cm⁻¹, then down to a curvature of K=−2.35 (±0.03) cm⁻¹, then back to the flat starting point of K=0 cm⁻¹. When finished, the results are graphically shown by the KES software. Save raw data from the test to a comma delimited text file, including the sample ID and MD in the name. This file can then be used for any analysis and calculations. Upon completion of the test, the instrument automatically loosens the jaws so the sample moves freely again. Pull the sample away from the jaws.

Next, test the CD of the same sample, by rotating the sample 90 degrees. Again, measure the width (this time in the MD direction) to the nearest 0.1 cm, at a distance approximately 1½ to 2½ inches from the sample end that will be fed into the instrument jaws (i.e., the end furthest from the person standing in front of the instrument). Record the distance (with respect to the sample ID). Slide the sample into the both jaws of the instrument, centered with relative to the jaw's width. When the sample is adequately positioned through both jaws, a small red light on the instrument illuminates to inform the tester that the test can begin. Press the MEASURE button—this will cause the instrument to automatically close the jaws, clamping the sample into place. Once the MEASURE button begins to blink on and off, then, using the KES software program, click the ‘Back’ button to begin a new test, provide a test name, and start the measurement. The instrument bends the sample as previously described. When finished, the results are graphically shown by the KES software. Save raw data from the test to a comma delimited text file, including the sample ID and CD in the name. This file is used later in analysis and calculations. Upon completion of the test, the instrument automatically loosens the jaws so the sample moves freely again. Pull the sample away from the jaws and discard. Repeat this procedure for the other 4 pre-cut uu test samples.

Next, a test is run with no sample in the instrument. This data will be used to remove the any noise inherent to the measurement system from the test sample measurement data. With nothing in the instrument jaws, a small piece of bond paper temporarily covers the red LED used to detect whether a sample is loaded within the jaws. This enables the instrument MEASURE button, when pressed, to begin closing the jaws and prepare for testing, just as if a sample were present in the instrument jaws. Once the jaws begin to close, the temporary cover on the LED light is removed. Once the MEASURE button begins to blink on and off, then, using the KES software program, click the ‘Back’ button to begin a new test, provide a test name, and start the measurement. The instrument moves the jaw as previously described. When finished, the results are graphically shown by the KES software. Save raw data from the test to a comma delimited text file, including the sample ID and “blank” in the name. This file is used later in analysis and calculations.

4. Calculations and Analysis

For each test condition, there are 11 data files: five for sample MD, 5 for the sample CD, and 1 for a ‘blank’ run. Each of these file includes the curvature position (K, in units of cm⁻¹) and bending moment per unit length (M, in units of g*cm/cm). Data is acquired (during testing) at a rate of about 10 points per second; thus, each file has roughly 189 data points recorded (±5).

Flexural rigidity is calculated by identifying the maximum and minimum curvature in the data array—the maximum and minimum curvature is between positive and negative 2.32 and 2.38 cm⁻¹, respectively. The average of the previous 4 data points just before maximum curvature (Kₘₐₓ₄) and moment (Mₘₐₓ₄), and the previous 4 data points just before minimum curvature (Kₘᵢₙ) and moment (Mₘᵢₙ₄) are then calculated. The uncorrected and un-normalized (for width) flexural rigidity (FRuu) is calculated as follows (units of g*cm²/cm):

FRuu=(Mₘₐₓ₄−Mₘᵢₙ₄)/(Kₘₐₓ₄−Kₘᵢₙ₄)

Recall from the instrument software set-up required the sample width to be a constant at 20 cm (W₂₀) even though the sample width is a variable that was manually measured with a ruler (W_{act}). The calculation for uncorrected flexural rigidity (FRu) is as foillows:

FRu=FRuu*W₂₀/W_{act}

The corrected and width normalized flexural rigidity (FR) is then calculated by subtracting the blank flexural rigidity normalized to 20 cm width (FRb), with FRb calculated in the same manner as described previously for FRuu.

FR=(FRuu−FRb)*W₂₀/W_{act}

This calculation process is performed for each of the 5 MD and 5 CD tests for a given sample condition. The results are then numerically averaged to produce a flexural rigidity for the MD (FR_{MD}) and CD (FR_{CD}), respectively. The average flexural rigidity (FR_{AVG}) for the sample condition is the numerical average of FR_{MD} and FR_{CD}.

### 9. Mellin Gauge (Dead Weight) Test Method

The dead weight compression tester (Mellin Gauge) is a manually test operated apparatus. The unit is used online as a quick reference tool to determine if finished packaged product is being produced at an optimum level. Readings can verify whether or not you are deviating to less than desired finished product results.

a. Overview:

Compressed stack height is determined using the Dead Weight Compression Tester (Mellin Gauge) to measure the compressed height of a stack of packaged tissue products by generally placing a known weight on top of the stack and measuring the stack height before and after application of the weight.

b. Equipment

Gauge Material:

All walls and base are of ½″ Lexan (clear) material.

All anchor points of assembly are done with 3/32″×1½ “self-tapping screws Channels are of aluminum

Gauge:

Base Plate & front plate: 12.5”×9.0″

Interior Channel Plates (2): 8″×12″

Set parallel @ 4⅜″ spacing intersecting to front plate at 90 degrees

¾″× 1/16″×12″ aluminum channel (2), set @ 2⅛″ mounted (e.g., super glue) on each interior channel wall as weight guide

Weight Block:
Material: Aluminum
Block 4¼″×2.0″×⅞″
3/16″ eyebolt with nut mounted in center of weight block

Caliper Gauge:

Thwing-Albert Instrument Company Philadelphia, Pa. Pro Gauge Part Number: 89-2012 110 Volt 60 cycle

The Mellin gauge is placed on a flat surface with the scale visible. 10 packaged tissue products are placed into the unit, stacked vertically. A 301.9 gram weight is placed in the center on the top package of the stack. Measurement is taken using the bottom edge of mass as a reference point. The scale reading (in mm) is taken and recorded.

c. On Line Sample Preparation:

Ten packaged tissue products are placed in a small carton container and allowed to condition for 30 minutes at 74° F.+/−2° F. and Humidity at 40%+/−3% before placing any sample product in the Mellin Gauge.

d. Process:

Packaged tissue product is placed and stacked on their face within the Mellin Gauge test apparatus one at a time so not to distort or crush the package. Place packaged product in tester lay flat face up, width facing front to back and length horizontal. The packages should be stacked neatly to provide consistent results. Once all 10 packs are placed within the test apparatus record the total stack height (in mm). A mass (301.9 grams) is positioned to be centered on the top package of the stack. After initial compression, the mass is allowed to remain upon the compressed stack for 15-20 seconds before reading the scale. The bottom edge of the mass is used as the reference point. Read the corresponding stack height (scale in millimeters). Record the resulting distance(s) (in mm) on a data sheet.

A new stack is configured for another reading at a different applied mass. The masses (in g) used are 300, 1300, 2300, 3300, 4300, 5300, 6300, 7300, and 8300. No replicate measurements are made upon a product stack once the product stack has been compressed at any mass. A new stack is used for each measurement taken.

e. Calculation:

The reported result is the initial measured stack height less the measured compressed stack height. All finished product should have a reading ranging between 220-250 millimeters as read on the gauge. If reading is not between 220-250 millimeters on the gauge, repeat the test with new product. Take individual caliper readings of each package of product (overall package height) using the caliper test method discussed supra. Compare the caliper results with the Chip/roll set results taken at the production facility. Chip/roll caliper target is preferably 25 mils with lower specified limit of 20 mils.

If caliper is out of specification reject chip/roll and take reading from new chip/roll before starting production.

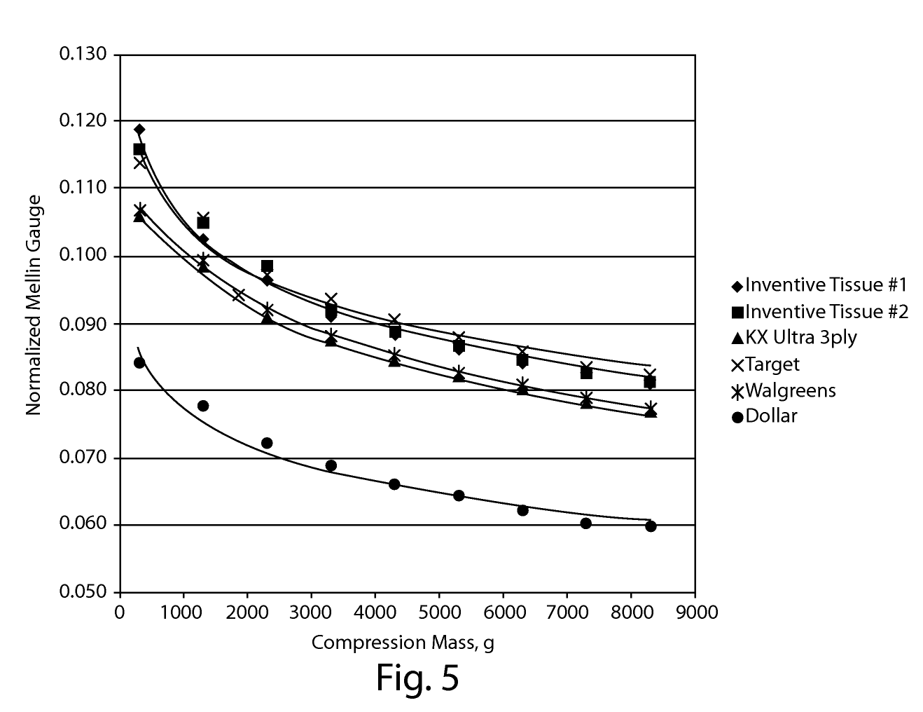

Normalized values are presented graphically in FIG. 5.

### Results

The products produced above in Examples 1 and 2, as well as several exemplary and commercially available products were tested using the test methods described supra. The results of this testing data are presented below in Table 1.

TABLE 1
Exemplary test results and data values for samples analyzed as discussed herein.

| | Total | | | | | Bulk | Bending |
|---|---|---|---|---|---|---|---|
| | Dry | Wet | | | Basis | Density | Flexibility |
| | Tensile | Burst | WB/TDT | COF - | Weight | @ 95 g/in² | (gf * cm²/cm) |
| Sample ID | (g/in) | (g) | ratio (in) | Dynamic | (g/m²) | (g/cm³) | (mg * cm²/cm) |
| Puffs Basic | 435 | 85 | 0.20 | 0.887 | 29 | 0.05 | 0.038 |
| Tempo | 1715 | 232 | 0.14 | | 64 | 0.07 | 0.186 |
| Puffs Ultra 07 | 727 | 137 | 0.19 | 0.922 | 37 | 0.07 | 0.048 |
| Kleenex | 470 | 42 | 0.09 | 1.017 | 29 | 0.07 | |
| Regular | | | | | | | |
| Kleenex Ultra | 577 | 66 | 0.11 | 0.880 | 43 | 0.05 | |
| Puff's Plus | 635 | 116 | 0.18 | 0.80 | 28 | 0.14 | 42.3 |
| Kleenex | 729 | 70 | 0.10 | | 26.5 | 0.19 | |
| Lotion 2007 | | | | | | | |
| Kleenex | 806 | 77 | 0.10 | | 29.5 | 0.13 | 10.1 |
| Lotion 2008 | | | | | | | |
| Example 1 | 530 | 105 | 0.20 | | 24 | | |
| Example 2 | 530 | 70 | 0.13 | | 23.5 | | |
| Example 3 | 530 | 125 | 0.24 | | 23.5 | | |

TABLE 2
Exemplary caliper test results and data values (in mils) for 10 replicate Example 1
packaged product samples produced and analyzed as discussed herein

| Pack #1 | 4 ply | DY Fold | Pack #2 | 4 ply | DY Fold | Pack #3 | 4 ply | DY Fold |
|---|---|---|---|---|---|---|---|---|
| Sheet 1 | 21.96 | 96.77 | Sheet 1 | 25.09 | 94.50 | Sheet 1 | 23.72 | 92.71 |
| Sheet 2 | 22.52 | 90.83 | Sheet 2 | 23.32 | 93.47 | Sheet 2 | 22.27 | 95.61 |
| Sheet 3 | 24.15 | 89.92 | Sheet 3 | 22.95 | 92.68 | Sheet 3 | 25.68 | 92.06 |
| Sheet 4 | 22.63 | 92.68 | Sheet 4 | 23.24 | 94.20 | Sheet 4 | 29.17 | 92.14 |
| Sheet 5 | 23.10 | 88.58 | Sheet 5 | 24.98 | 93.17 | Sheet 5 | 25.28 | 90.13 |
| Sheet 6 | 23.39 | 92.41 | Sheet 6 | 24.26 | 94.99 | Sheet 6 | 22.89 | 95.34 |
| Sheet 7 | 23.27 | 91.34 | Sheet 7 | 23.56 | 93.19 | Sheet 7 | 24.37 | 95.02 |
| Sheet 8 | 22.84 | 90.83 | Sheet 8 | 22.15 | 91.45 | Sheet 8 | 22.44 | 91.56 |
| Sheet 9 | 21.21 | 91.48 | Sheet 9 | 23.40 | 91.58 | Sheet 9 | 23.57 | 90.64 |
| Sheet 10 | 22.36 | 92.87 | Sheet 10 | 22.99 | 91.70 | Sheet 10 | 22.33 | 93.24 |
| 10 ct | 30 mm | | 10 ct | 30 mm | | 10 ct | 30 mm | |
| height | | | height | | | height | | |
| Average | 22.74 | 91.77 | Average | 23.59 | 93.09 | Average | 24.17 | 92.85 |
| Pack #4 | 4 ply | DY Fold | Pack #5 | 4 ply | DY Fold | Pack #6 | 4 ply | DY Fold |
| Sheet 1 | 24.19 | 92.00 | Sheet 1 | 25.49 | 96.61 | Sheet 1 | 23.10 | 90.48 |
| Sheet 2 | 22.97 | 90.58 | Sheet 2 | 22.99 | 90.34 | Sheet 2 | 24.02 | 93.65 |
| Sheet 3 | 33.54 | 90.68 | Sheet 3 | 24.56 | 91.37 | Sheet 3 | 22.80 | 91.38 |
| Sheet 4 | 23.76 | 93.50 | Sheet 4 | 23.74 | 91.53 | Sheet 4 | 22.41 | 94.26 |
| Sheet 5 | 23.79 | 93.13 | Sheet 5 | 24.24 | 92.69 | Sheet 5 | 25.61 | 95.22 |
| Sheet 6 | 29.94 | 96.60 | Sheet 6 | 23.29 | 90.63 | Sheet 6 | 22.64 | 94.16 |
| Sheet 7 | 35.02 | 94.32 | Sheet 7 | 26.57 | 89.42 | Sheet 7 | 23.87 | 90.76 |
| Sheet 8 | 24.21 | 92.54 | Sheet 8 | 23.48 | 92.55 | Sheet 8 | 22.18 | 92.03 |
| Sheet 9 | 29.50 | 99.35 | Sheet 9 | 25.83 | 93.40 | Sheet 9 | 23.36 | 90.73 |
| Sheet 10 | 23.07 | 98.98 | Sheet 10 | 26.72 | 88.27 | Sheet 10 | 23.02 | 89.68 |
| 10 ct | 32 mm | | 10 ct | 32 mm | | 10 ct | 32 mm | |
| height | | | height | | | height | | |
| Average | 27.00 | 94.17 | Average | 24.69 | 91.68 | Average | 23.30 | 92.24 |

| Pack #7 | 4 ply | DY Fold | Pack #8 | 4 ply | DY Fold | Pack #9 | DY Fold | 4 ply |
|---|---|---|---|---|---|---|---|---|
| Sheet 1 | 22.53 | 91.54 | Sheet 1 | 31.92 | 91.28 | Sheet 1 | 24.95 | 93.05 |
| Sheet 2 | 24.32 | 88.20 | Sheet 2 | 22.96 | 91.53 | Sheet 2 | 23.71 | 90.46 |
| Sheet 3 | 24.66 | 91.65 | Sheet 3 | 23.43 | 96.18 | Sheet 3 | 24.05 | 92.41 |
| Sheet 4 | 22.99 | 94.31 | Sheet 4 | 23.82 | 91.49 | Sheet 4 | 22.02 | 90.52 |
| Sheet 5 | 22.95 | 91.37 | Sheet 5 | 25.77 | 94.63 | Sheet 5 | 35.10 | 91.97 |
| Sheet 6 | 23.16 | 90.13 | Sheet 6 | 29.02 | 90.70 | Sheet 6 | 30.32 | 94.80 |
| Sheet 7 | 21.78 | 94.06 | Sheet 7 | 24.19 | 97.67 | Sheet 7 | 26.25 | 96.01 |
| Sheet 8 | 22.17 | 92.51 | Sheet 8 | 23.39 | 92.75 | Sheet 8 | 27.31 | 91.69 |
| Sheet 9 | 21.91 | 97.71 | Sheet 9 | 22.50 | 93.37 | Sheet 9 | 33.81 | 89.21 |
| Sheet 10 | 23.52 | 90.36 | Sheet 10 | 22.85 | 99.71 | Sheet 10 | 32.95 | 95.54 |

| 10 ct | 31 mm | | 10 ct | 34 mm | | 10 ct | 32 mm | |
|---|---|---|---|---|---|---|---|---|
| height | | | height | | | height | | |
| Average | 23.00 | 92.18 | Average | 24.99 | 93.93 | Average | 28.05 | 92.57 |

| Pack #10 | DY Fold | 4 ply |
|---|---|---|
| Sheet 1 | 27.94 | 93.94 |
| Sheet 2 | 23.91 | 90.26 |
| Sheet 3 | 24.99 | 88.50 |
| Sheet 4 | 27.73 | 94.92 |
| Sheet 5 | 22.11 | 94.00 |
| Sheet 6 | 25.01 | 91.92 |
| Sheet 7 | 23.03 | 88.88 |
| Sheet 8 | 24.74 | 94.35 |
| Sheet 9 | 22.34 | 90.98 |
| Sheet 10 | 23.19 | 92.93 |

| | |
|---|---|
| 10 ct | 34 mm |
| height | |

| | | |
|---|---|---|
| Average | 24.50 | 92.07 |

TABLE 3
Exemplary Mellin Gauge values for samples analyzed as discussed herein.

| | | Sample ID | Sample ID | | | |
|---|---|---|---|---|---|---|
| | Sample ID | Target To Go | Walgreen To Go | Sample ID | Sample ID | Sample ID |
| | Kleenex ToGo | 15 ct 2-ply | 15 ct 2-ply | Dollar Tree To Go | Inventive | Inventive |
| | 10 ct 3-ply | unscented | unscented | 10 ct 2-ply | Tissue #1 | Tissue #2 |
| | (unlotioned) | (unlotioned) | (unlotioned) | (unlotioned) | (lotioned) | (lotioned) |
| | Single Tissue | Single Tissue | Single Tissue | Single Tissue | Single Tissue | Single Tissue |
| | 4-ply Average | 4-ply Average | 4-ply Average | 4-ply Average | 4-ply Average | 4-ply Average |
| | Caliper (mm) | Caliper (mm) | Caliper (mm) | Caliper (mm) | Caliper (mm) | Caliper (mm) |
| | 23.87 | 15.41 | 15.27 | 12.24 | 19.57 | 19.95 |
| Applied | Mellin Gauge | Mellin Gauge | Mellin Gauge | Mellin Gauge | Mellin Gauge | Mellin Gauge |
| Mass | 10 pack avg | 10 pack avg | 10 pack avg | 10 pack avg | 10 pack avg | 10 pack avg |
| (g) | (in) | (in) | (in) | (in) | (in) | (in) |
| 300 | 25.28 | 26.33 | 24.48 | 10.30 | 23.2 | 23.1 |
| 1300 | 23.54 | 24.42 | 22.82 | 9.52 | 20.1 | 21.0 |
| 2300 | 21.76 | 22.52 | 21.12 | 8.86 | 18.9 | 19.7 |
| 3300 | 20.90 | 21.74 | 20.26 | 8.46 | 17.9 | 18.4 |
| 4300 | 20.20 | 21.02 | 19.58 | 8.12 | 17.4 | 17.8 |
| 5300 | 19.66 | 20.42 | 18.98 | 7.92 | 16.9 | 17.4 |
| 6300 | 19.20 | 19.92 | 18.60 | 7.66 | 16.5 | 16.9 |
| 7300 | 18.74 | 19.38 | 18.12 | 7.42 | 16.3 | 16.5 |
| 8300 | 18.44 | 19.10 | 17.78 | 7.36 | 15.9 | 16.3 |

TABLE 4
Mellin Gauge Values from Table 3 Normalized by Caliper.

| Applied | Inventive | Inventive | Kleenex To Go | Target To Go | Walgreen To Go | Dollar Tree To Go |
|---|---|---|---|---|---|---|
| Mass (g) | Tissue #1 | Tissue #2 | 3-ply | 2-ply | 2-ply | 2-ply |
| 300 | 1.188 | 1.158 | 1.059 | 1.707 | 1.603 | 0.842 |
| 1300 | 1.026 | 1.051 | 0.986 | 1.585 | 1.494 | 0.778 |
| 2300 | 0.966 | 0.987 | 0.912 | 1.461 | 1.383 | 0.724 |
| 3300 | 0.913 | 0.922 | 0.876 | 1.411 | 1.327 | 0.691 |
| 4300 | 0.887 | 0.890 | 0.846 | 1.364 | 1.282 | 0.663 |
| 5300 | 0.866 | 0.870 | 0.824 | 1.325 | 1.243 | 0.647 |
| 6300 | 0.844 | 0.849 | 0.804 | 1.293 | 1.218 | 0.626 |
| 7300 | 0.831 | 0.829 | 0.785 | 1.258 | 1.187 | 0.606 |
| 8300 | 0.813 | 0.817 | 0.773 | 1.239 | 1.164 | 0.601 |
| ct | 10 | 10 | 10 | 15 | 15 | 10 |

TABLE 5
Mellin Gauge Values from Table 3 Normalized by Caliper and Number of
Sheets/Package.

| Applied | Inventive | Inventive | KX To Go | Target To Go | Walgreens To Go | Dollar To Go |
|---|---|---|---|---|---|---|
| Mass (g) | Tissue #1 | Tissue #2 | 3ply | 3-ply | 2-ply | 2-ply |
| 300 | 0.119 | 0.116 | 0.106 | 0.114 | 0.107 | 0.084 |
| 1300 | 0.103 | 0.105 | 0.099 | 0.106 | 0.100 | 0.078 |
| 2300 | 0.097 | 0.099 | 0.091 | 0.097 | 0.092 | 0.072 |
| 3300 | 0.091 | 0.092 | 0.088 | 0.094 | 0.088 | 0.069 |
| 4300 | 0.089 | 0.089 | 0.085 | 0.091 | 0.085 | 0.066 |
| 5300 | 0.087 | 0.087 | 0.082 | 0.088 | 0.083 | 0.065 |
| 6300 | 0.084 | 0.085 | 0.080 | 0.086 | 0.081 | 0.063 |
| 7300 | 0.083 | 0.083 | 0.079 | 0.084 | 0.079 | 0.061 |
| 8300 | 0.081 | 0.082 | 0.077 | 0.083 | 0.078 | 0.060 |

TABLE 6
Mathematically Expressed Equations Folded and Packaged
Facial Tissue Products Where x = Applied Mass
and y = Normalized Mellin Gauge (by caliper) Value.

| | |
|---|---|
| Kleenex To Go: | y = −0.009ln(x) + 0.1598 R² = 0.9692 |
| Target To Go: | y = −0.010ln(x) + 0.1720 R² = 0.9704 |
| Walgreen To Go: | y = −0.009ln(x) + 0.1619 R² = 0.9663 |
| Dollar Tree To Go: | y = −0.008ln(x) + 0.1297 R² = 0.9655 |
| Inventive Tissue #1: | y = −0.011ln(x) + 0.1834 R² = 0.9992 |
| Inventive Tissue #2: | y = −0.011ln(x) + 0.1792 R² = 0.9799 |

A preferred embodiment of the present invention provides a wet burst value of greater than about 80 grams, preferably ranges from about 90 grams to 400 grams, more preferably ranges from about 100 grams to about 200 grams. A preferred embodiment of the product of the present invention provides a dynamic coefficient of friction value of less than about 0.9, preferably ranging from about 0.6 to about 0.9, more preferably ranges from about 0.6 to about 0.85, and even more preferably ranges from about 0.75 to about 0.85. A preferred embodiment of a product of the present invention having lotion applied thereto provides a bending flexibility of less than about 50 mg*cm²/cm, preferably ranges from about 5 mg*cm²/cm to about 30 mg*cm²/cm, and more preferably ranges from about 10 mg*cm²/cm to about 21 mg*cm²/cm. A preferred embodiment of the present invention provides a wet burst/total dry tensile ratio value of greater than about 0.12 inches, preferably ranges from about 0.14 inches to about 0.30 inches, and more preferably ranges from about 0.16 inches to about 0.24 inches.

The dimensions and values disclosed herein are not to be understood as being strictly limited to the exact dimension and values recited. Instead, unless otherwise specified, each such dimension and/or value is intended to mean both the recited dimension and/or value and a functionally equivalent range surrounding that dimension and/or value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A stack of folded web substrates each folded web substrate of said stack of folded web substrates having a folded surface area of about A/8 and being produced from a respective unfolded web substrate having an unfolded surface area of A and having a lotion applied to one surface thereof, said stack of folded web substrates having a normalized Mellin gauge value of greater than 0.06.

2. The stack of folded web substrates of claim 1 wherein said lotion comprises:
a. From about 10 percent to about 90 percent of a compound selected from the group consisting of oils, emollients, and waxes; and,
b. Less than about 20 percent of water content.

3. The stack of folded web substrates of claim 2 wherein said lotion composition further comprises from at least about 15 percent to about 100 percent solids content.

4. The stack of folded web substrates of claim 1 wherein each of said unfolded web substrates further comprises a chemical softening agent.

5. The stack of folded web substrates of claim 1 wherein each of said unfolded web substrates each comprises from about 2.0 percent to about 25.0 percent of said lotion based upon a dry fiber weight of said web substrate.

6. The stack of folded web substrates of claim 5 wherein each of said unfolded web substrates each comprises from about 4.0 percent to about 11.0 percent of said lotion based on the dry fiber weight of said unfolded web substrate.

7. The stack of folded web substrates of claim 1 wherein said lotion comprises a compound selected from the group consisting of glycols, polyglycols, petrolatum, fatty acids, fatty alcohols, fatty alcohol ethoxylates, fatty alcohol esters and fatty alcohol ethers, fatty acid ethoxylates, fatty acid amides and fatty acid esters, hydrocarbon oils (such as mineral oil), squalane, fluorinated emollients, silicone oil, and mixtures thereof.

8. The stack of folded web substrates of claim 1 wherein said lotion comprises an emollient selected from the group consisting of petroleum-based emollients, fatty acid ester type emollients, alkyl ethoxylate type emollients, and combinations thereof.

9. The stack of folded web substrates of claim 1, wherein each of said unfolded web substrates are creped.

10. A stack of folded web substrates each having a folded surface area of about A/8 each produced from a respective un-folded web substrate having an unfolded surface area of A and having a lotion applied to one surface thereof, said stack of folded web substrates having a normalized Mellin gauge value satisfying the equation y≧−0.008 ln(x)+0.1297 where x=applied mass and y=normalized Mellin Gauge (by caliper) value.

11. The stack of folded web substrates of claim 10 wherein said lotion comprises:
a. From about 10 percent to about 90 percent of a compound selected from the group consisting of oils, emollients, and waxes; and,
b. Less than about 20 percent of water content.

12. The stack of folded web substrates of claim 11 wherein the lotion composition further comprises from at least about 15 percent to about 100 percent solids content.

13. The stack of folded web substrates of claim 10 wherein said Mellin Gauge value is greater than about 0.06.

14. The stack of folded web substrates of claim 10 wherein said unfolded web substrates each comprise from about 2.0 percent to about 25.0 percent of said lotion based upon a dry fiber weight of said unfolded web substrate.

15. The stack of folded web substrates of claim 14 wherein said unfolded web substrates each comprises from about 4.0 percent to about 11.0 percent of said lotion based on the dry fiber weight of said unfolded web substrate.

16. The stack of folded web substrates of claim 10 wherein said lotion comprises a compound selected from the group consisting of glycols, polyglycols, petrolatum, fatty acids, fatty alcohols, fatty alcohol ethoxylates, fatty alcohol esters and fatty alcohol ethers, fatty acid ethoxylates, fatty acid amides and fatty acid esters, hydrocarbon oils (such as mineral oil), squalane, fluorinated emollients, silicone oil, and mixtures thereof.

17. The stack of folded web substrates of claim 10 wherein said lotion comprises an emollient selected from the group consisting of petroleum-based emollients, fatty acid ester type emollients, alkyl ethoxylate type emollients, and combinations thereof.

18. The stack of folded web substrates of claim 10, wherein each of said unfolded paper products are creped.
